(19) United States
(12) Patent Application Publication
(10) Pub. No.: US 2012/0083694 A1
(43) Pub. Date: Apr. 5, 2012

(54) STRETCHING BASED STRAIN IMAGING IN AN ULTRASOUND SYSTEM

(75) Inventors: Mok Kun JEONG, Seoul (KR); Sang Shik Park, Seoul (KR); Sung Jae Kwon, Seoul (KR)

(73) Assignee: MEDISON CO., LTD.

(21) Appl. No.: 13/075,972

(22) Filed: Mar. 30, 2011

(30) Foreign Application Priority Data
Oct. 1, 2010 (KR) 10-2010-0095702

(51) Int. Cl. A61B 8/00

(52) U.S. Cl. 600/438

(57) ABSTRACT

Embodiments for forming an elastic image in an ultrasound system are disclosed. In one embodiment, a processing unit is configured to control an ultrasound data acquisition unit to perform the transmit/receive operation in a first state where compression is not applied to the target object to thereby obtain a first ultrasound frame data set, and to perform the transmit/receive operation in a second state where compression is applied to the target object to thereby obtain a second ultrasound frame data set. The processing unit is further configured to compute a first strain between the first and second ultrasound frame data sets, perform globally uniform stretching upon the second ultrasound frame data set based on the first strain, and compute a second strain between the first ultrasound frame data set and the stretched second ultrasound frame data set and form an elastic image based on the second strain.

### CROSS-REFERENCE TO RELATED APPLICATIONS

The present application claims priority from Korean Patent Application No. 10-2010-0095702 filed on Oct. 1, 2010, the entire subject matter of which is incorporated herein by reference.

### TECHNICAL FIELD

The present disclosure generally relates to strain imaging, and more particularly to stretching based strain imaging in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two or three-dimensional images of internal features of an object (e.g., human organs).

Generally, the ultrasound image is displayed in a Brightness-mode (B-mode) by using reflectivity caused by an acoustic impedance difference between the tissues of the target object. However, if the reflectivity of the target object is hardly different from those of the neighboring tissues such as tumor, cancer or the like, then it is not easy to recognize the target object in the B-mode image.

To cope with the problem of recognizing the tumor, cancer and the like in the B-mode, an ultrasound elasticity imaging has been developed to visualize the mechanical characteristics of the tissues such as the elasticity of the same in the ultrasound system. Such imaging was proven to be very helpful for diagnosing lesions such as tumor and cancer, which would otherwise be hardly recognized in the B-mode image, in soft tissues (e.g., breast). The ultrasound elasticity imaging may utilize the scientific property that the elasticity of the tissues is related to a pathological phenomenon. For example, the tumor or cancer is relatively stiffer than the surrounding normal tissues. Thus, when stress is uniformly applied, a strain of the tumor or cancer may be typically smaller than those of the surrounding tissues. When a strain image visualizing the strains measured in the target object (also called an “elastic image”) is displayed on a display unit, relatively stiff portions may be indicated darkly and relatively soft portions may be indicated brightly for visual recognition. Thus, since the stiff portion at which lesion or cancer exists is displayed darkly, a contrast therein may be lowered.

### SUMMARY

Embodiments for forming a strain image with enhanced contrast at hard regions of a target object in an ultrasound system are disclosed herein. In one embodiment, by way of non-limiting example, an ultrasound system may comprise: an ultrasound data acquisition unit configured to perform a transmit/receive operation including transmitting ultrasound signals to a target object and receiving ultrasound echoes reflected from the target object to thereby acquire ultrasound frame data; and a processing unit configured to control the ultrasound data acquisition unit to perform the transmit/receive operation in a first state where compression is not applied to the target object to thereby obtain a first ultrasound frame data set, the processing unit being further configured to control the ultrasound data acquisition unit to perform the transmit/receive operation in a second state where compression is applied to the target object to thereby obtain a second ultrasound frame data set, the processing unit being configured to compute a first strain between the first and second ultrasound frame data sets and perform globally uniform stretching upon the second ultrasound frame data set based on the first strain, the processing unit being further configured to compute a second strain between the first ultrasound frame data set and the stretched second ultrasound frame data set and form an elastic image based on the second strain.

In another embodiment, a method of forming a strain image in an ultrasound system, may comprise: a) acquiring first ultrasound frame data set from a target object without applying compression to the target object; b) acquiring second ultrasound frame data set from a target object with applying compression to the target object; c) computing a first strain between the first and second ultrasound frame data set; d) performing globally uniform stretching upon the second ultrasound frame data set based on the first strain; e) computing a second strain between the first ultrasound frame data set and the stretched second ultrasound frame data set; and f) forming a strain image based on the second strain.

In yet another embodiment, a computer-readable storage medium storing instructions that, when executed by a computer, cause the computer to provide a method of forming an elastic image based on first ultrasound frame data set acquired from a target object without applying compression to the target object and second ultrasound frame data set acquired from the target object with applying compression to the target object in an ultrasound system is provided, wherein the method comprises: computing a first strain between the first and second ultrasound frame data set; performing globally uniform stretching upon the second ultrasound frame data set based on the first strain; computing a second strain between the first ultrasound frame data set and the stretched second ultrasound frame data set; and forming a strain image based on the second strain.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a block diagram showing an illustrative embodiment of an ultrasound data acquisition unit of FIG. 1.

FIG. 3 is a flowchart showing an illustrative embodiment of forming a strain image.

FIG. 4 is a schematic diagram showing an example of performing globally uniform stretching upon ultrasound data acquired from a target object with applying compression to the target object.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

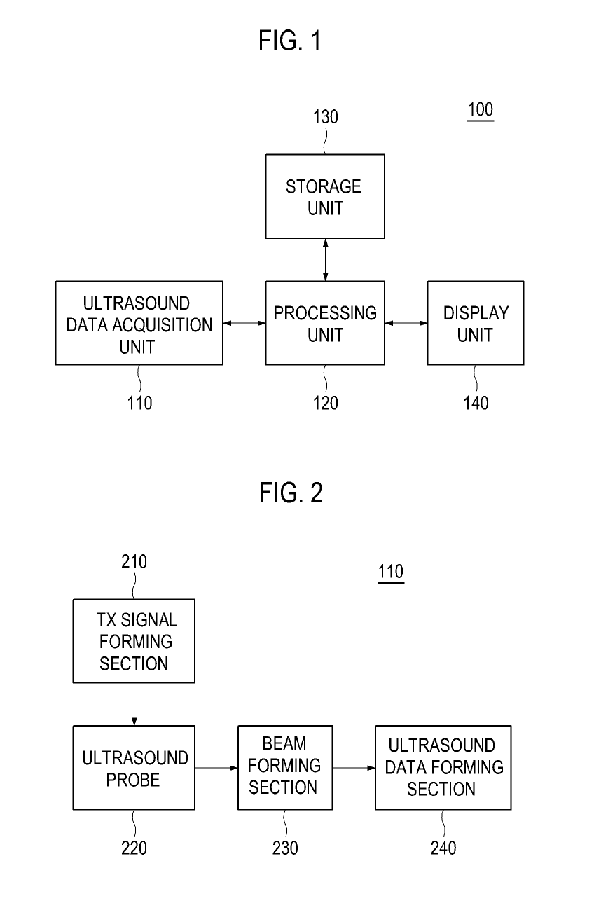

Referring to FIG. 1, an ultrasound system constructed in accordance with one embodiment is shown. The ultrasound system 100 may include an ultrasound data acquisition unit 110, a processing unit 120, a storage unit 130 and a display unit 140.

The ultrasound data acquisition unit 110 may be configured to transmit ultrasound beams to a target object and receive ultrasound echoes reflected from the target object to thereby form ultrasound data representative of the target object. An operation of the ultrasound acquisition unit will be described in detail by referring to FIG. 2.

FIG. 2 is a block diagram showing an illustrative embodiment of the ultrasound data acquisition unit 110. Referring to FIG. 2, the ultrasound data acquisition unit 110 may include a transmit (Tx) signal forming section 210. The Tx signal forming section 210 may generate a plurality of Tx signals and apply delays to the Tx signals.

The ultrasound data acquisition unit 110 may further include an ultrasound probe 220, which is coupled to the Tx signal forming section 210. The ultrasound probe 220 may include an array transducer containing a plurality of transducer elements for reciprocal conversion between electric signals and ultrasound signals. The ultrasound probe 220 may be configured to transmit ultrasound signals in response to the Tx signals. The ultrasound probe 220 may be further configured to receive ultrasound echoes reflected from the target object to thereby output receive signals. In one embodiment, the receive signals may include first receive signals obtained without applying compression to the target object and second receive signals obtained with applying compression to the target object.

The ultrasound data acquisition unit 110 may further include a beam forming section 230, which is coupled to the ultrasound probe 220. The beam forming section 230 may be configured to digitize the electrical receive signals into digital signals. The beam forming section 230 may also apply delays to the digital signals in consideration of distances between the elements of the ultrasound probe 220 and focal points. The beam forming section 230 may further sum the delayed digital signals to form receive-focused signals. In one embodiment, the beam forming section 230 may form first receive-focused signals based on the first receive signals and second receive-focused signals based on the second receive signals.

The ultrasound data acquisition unit 110 may further include an ultrasound data forming section 240, which is coupled to the beam forming section 230. The ultrasound data forming section 240 may be configured to form ultrasound frame data sets corresponding to a plurality of frames based on the receive-focused signals. The ultrasound frame data sets may include RF data sets or in-phase/quadrature (IQ) data sets. However, the ultrasound data may not be limited thereto. The ultrasound data forming section 240 may be further configured to perform a variety of signal processing (e.g., gain adjustment, filtering, etc.) upon the receive-focused signals. In one embodiment, the ultrasound data may include a first ultrasound frame data set formed based on the first receive-focused signals and a second ultrasound frame data set formed based on the second receive-focused signals.

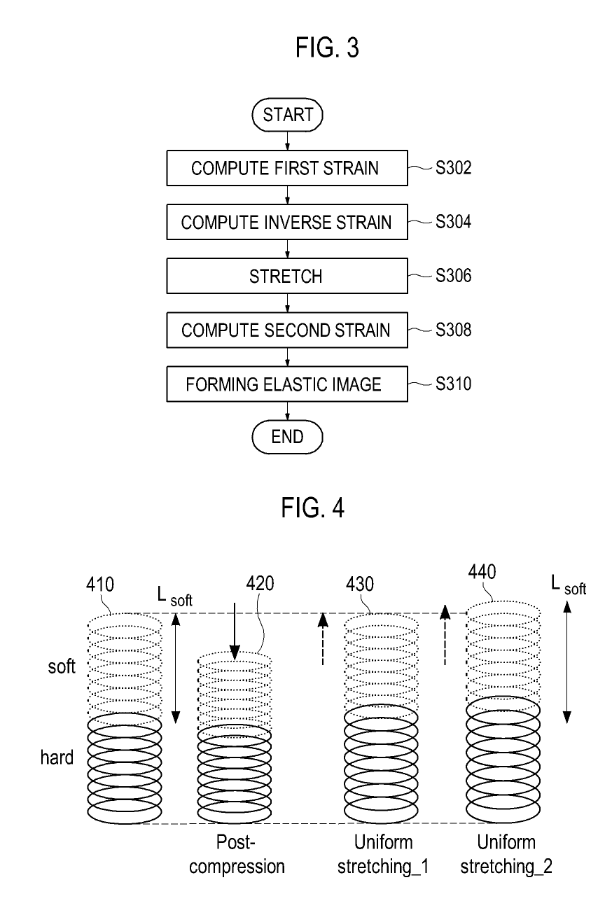

Referring back to FIG. 1, the processing unit 120, which is coupled to the ultrasound data acquisition unit 110, may be embodied with at least one of a central processing unit, a microprocessor, a graphic processing unit and the like. However, the processing unit 120 may not be limited thereto. The processing unit 120 may be configured to control the ultrasound data acquisition unit 110 to perform the transmit/receive operation in a first state where compression is not applied to the target object to thereby obtain the first ultrasound frame data set. Further, the processing unit 120 may be configured to control the ultrasound data acquisition unit 110 to perform the transmit/receive operation in a second state where compression is applied to the target object to thereby obtain the second ultrasound frame data set. The processing unit 120 may be configured to form a strain image (i.e., elastic image) based on the first and second ultrasound frame data sets. The formation process of the strain image will be described in detail by referring to FIG. 3.

FIG. 3 is a flowchart showing an illustrative embodiment of forming the strain image. Referring to FIG. 3, the processing unit 120 may be configured to compute strains between the first ultrasound frame data set and the second ultrasound frame data set at S302. The ultrasound frame data set obtained without applying compression to the target object, i.e., the first ultrasound frame data set, and the ultrasound frame data set obtained with applying compression to the target object, i.e., the second ultrasound frame data set, are defined by the following equations for modeling the compression of medium within the target object.

xₚᵣₑ(t)=r(t)

xₚₒₛₜ(t)=r(at) (1)

wherein xₚᵣₑ(t) represents the first ultrasound frame data set, xₚₒₛₜ(t) represents the second ultrasound frame data set, r(t) and r(at) represent envelopes of the first and second frame data sets and a represents a compression coefficient for scaling in the time axis.

Assuming that a length of the medium without the compression applied is L₀ and a length of the medium with the compression applied is L, a strain s and the compression coefficient a may be computed by the following equations.

$$s = \frac{{L}_{0} - L}{{L}_{0}} \\ a = \frac{1}{1 - s} ≅ 1 + s . \qquad ( 2 )$$

Thus, the strain s and the compression coefficient a for the soft medium (e.g., soft tissues) and the hard medium (e.g., tumor, cancer, etc.) may be represented by the following equations.

s_{soft}>s_{hard}

a_{soft}>a_{hard} (3)

wherein s_{soft} represents a strain of the soft medium, s_{hard} represent a strain of the hard medium, a_{soft} represents a compression coefficient of the soft medium and a_{hard} represents a compression coefficient of the hard medium. Thus, if the target object is compressed, then s>0 and a>1, and if the target object is stretched, then s<0 and a<1.

The processing unit 120 may be further configured to compute a strain s with which to perform globally uniform stretching (hereinafter referred to as “reverse strain”) at S304. The second ultrasound frame data set may be defined by the following equations that model the compression of medium.

x_{post-soft}(t)=r(a_{soft}t),

x_{post-hard}(t)=r(a_{hard}t) (4)

wherein x_{post soft}(t) represents the second ultrasound frame data set corresponding to the soft medium and x_{post hard}(t) represents the second ultrasound frame data set corresponding to the hard medium.

If the second ultrasound frame data set x_{post-soft}(t) and x_{post hard}(t) are stretched by a compression coefficient a_{global}, then the second ultrasound frame data set may be expressed by the following equations.

x_{post-soft-global}(t)=r(a_{soft}·a_{global}t),

x_{post-hard-global}(t)=r(a_{hard}·a_{global}t) (5)

wherein x_{post-hard-global}(t) represents an ultrasound frame data set obtained by stretching the second ultrasound frame data set x_{post-soft}(t) corresponding to the soft medium by the compression coefficient a_{global} and x_{post-hard-global}(t) represents an ultrasound frame data set obtained by stretching the second ultrasound frame data set x_{post-hard}(t) corresponding to the hard medium by the compression coefficient a_{global}.

Thus, with the adjustment of the compression coefficient a_{global}, an identical effect to compressing the medium in the target object by different coefficients may be achieved. If the compression coefficient a_{global} is adjusted such that a_{soft}·a_{global}=1 then the soft medium may be indicated as if the compression was not applied and the hard medium may be indicated as being stretched due to the relationship a_{hard}·a_{global}<1<a_{hard}. As such, hardness and softness may be inversely indicated in the strain image compared to the conventional strain image.

FIG. 4 is a schematic diagram showing an example of performing globally uniform stretching upon the second ultrasound frame data set. Referring to FIG. 4, if the globally uniform stretching is performed upon the second ultrasound frame data set, then the soft medium, which is relatively more prone to deformation, may be restored to an original state. However, the hard medium, which is relatively less to deformation, may be more stretched than an original state.

That is, after the process, which renders the stretching process with an adjusted degree of the globally uniform stretching, to restore the soft medium to the original state, if the strain is computed according to the conventional elasticity imaging, then the hard medium may be indicated as a soft region in the strain image. When a length of the soft medium in the first ultrasound frame data set 410 obtained without the compression applied is L_{soft} (see FIG. 4), the second ultrasound frame data set 420 obtained with the compression applied should be stretched by a reverse strain greater than a strain present in the second ultrasound frame data set to restore the length of the soft medium. Reference numeral “430” in FIG. 4 represents the second ultrasound frame data set, which is stretched by a reverse strain identical to the strain caused in the second ultrasound frame data set, while reference numeral “440” represents the second ultrasound frame data set stretched by a reverse strain greater than the strain present in the second ultrasound frame data set and to the degree that a length of the soft medium becomes L_{soft}.

Referring back to FIG. 3, the processing unit 120 may be further configured to perform the globally uniform stretching upon the second ultrasound frame data set based on the reverse strain to thereby form globally and uniformly stretched second ultrasound frame data set at S306. The processing unit 120 may be configured to compute a strain between the first ultrasound frame data set and the stretched second ultrasound frame data set at S308, and form an elastic image by using the computed strain at S310.

The storage unit 130, which is coupled to the ultrasound data acquisition unit 110 via the processing unit 120, is configured to store the ultrasound data acquired in the ultrasound data acquisition unit 110. Also, the storage unit 130 may be configured to store the computed strain. The display unit 140 may display the elastic image, which has been formed in the processing unit 120. The display unit 140 may include at least one of a cathode ray tube (CRT) display, a liquid crystal display (LCD), an organic light emitting diode (OLED) display and the like.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an ultrasound data acquisition unit configured to perform a transmit/receive operation including transmitting ultrasound signals to a target object and receiving ultrasound echoes reflected from the target object to thereby acquire ultrasound frame data; and
a processing unit configured to control the ultrasound data acquisition unit to perform the transmit/receive operation in a first state where compression is not applied to the target object to thereby obtain a first ultrasound frame data set and control the ultrasound data acquisition unit to perform the transmit/receive operation in a second state where compression is applied to the target object to thereby obtain a second ultrasound frame data set, the processing unit being configured to compute a first strain between the first and second ultrasound frame data sets and perform globally uniform stretching upon the second ultrasound frame data set based on the first strain, the processing unit being further configured to compute a second strain between the first ultrasound frame data set and the stretched second ultrasound frame data set and form an elastic image based on the second strain.

2. The ultrasound system of claim 1, wherein the processing unit is further configured to compute a reverse strain for the globally uniform stretching based on the first strain and perform the globally uniform stretching upon the second ultrasound frame data set based on the reverse strain.

3. A method of forming an elastic image in an ultrasound system, comprising:
a) acquiring first ultrasound frame data set from a target object without applying compression to the target object;
b) acquiring second ultrasound frame data set from a target object with applying compression to the target object;
c) computing a first strain between the first and second ultrasound frame data sets;
d) performing globally uniform stretching upon the second ultrasound frame data set based on the first strain;
e) computing a second strain between the first ultrasound frame data set and the stretched second ultrasound frame data set; and
f) forming an elastic image based on the second strain.

4. The method of claim 3, wherein the step d) includes:
computing a reverse strain for the globally uniform stretching based on the first strain; and
performing the globally uniform stretching upon the second ultrasound frame data set based on the reverse strain.

5. A computer-readable storage medium storing instructions that, when executed by a computer, cause the computer to provide a method of forming an elastic image based on first ultrasound frame data set acquired from a target object without applying compression to the target object and second ultrasound frame data set acquired from the target object with applying compression to the target object in an ultrasound system, the method comprising:
computing a first strain between the first and second ultrasound frame data sets;
performing globally uniform stretching upon the second ultrasound frame data set based on the first strain;
computing a second strain between the first ultrasound frame data set and the stretched second ultrasound frame data set; and
forming an elastic image based on the second strain.
